(19) United States
(12) Patent Application Publication
(10) Pub. No.: US 2005/0222598 A1
(43) Pub. Date: Oct. 6, 2005

(54) Tissue cutting device

(75) Inventors: Huddee Jacob Ho, San Jose, CA (US); Roberta Lee, Redwood City, CA (US); Samuel E. Zuckswert, San Jose, CA (US)

Correspondence Address: Jung-hua Kuo;Attorney At Law, PO Box 3275, Los Altos, CA, 94024, US

(73) Assignee: MANOA MEDICAL, INC., A DELAWARE CORPORATION, Redwood City, CA (US)

(21) Appl. No.: 10/819,083

(22) Filed: Apr. 5, 2004

(51) Int. Cl. A61B 17/32

(52) U.S. Cl. 606/171; 606/167

(57) ABSTRACT

Devices for efficient severing or cutting of a material or substance such as soft tissue suitable for use in open surgical and/or minimally invasive procedures are disclosed. A cutting assembly generally includes a first and second cutting blades each having an inner surface and at least one set of cutting teeth, the first inner surface being in contact with the second inner surface so that the sets of cutting teeth of the first and second cutting blades are aligned with and configured to cooperate with each other when at least one of the cutting blades moves, e.g., rotates and/or oscillates, relative to the other. A tissue cutting device generally includes a probe and the cutting assembly configured to be in a storage configuration or in a cutting configuration. The cutting assembly may provide a coagulation mechanism.

### CROSS REFERENCE TO RELATED APPLICATIONS

This application is related to co-pending U.S. patent application Ser. No. 10/______ (Attorney Docket No. MNOAP008), entitled “Tissue Cutting Devices and Methods” and filed on Mar. 31, 2004, the entirety of which is incorporated by reference herein.

### BACKGROUND OF THE INVENTION

1. Field of the Invention

The present invention relates generally to devices for cutting a material or substance. More specifically, devices for efficient severing or cutting of a material or substance such as soft tissue suitable for use in open surgical and/or minimally invasive procedures are disclosed.

2. Description of Related Art

Standard methods of severing of tissue may include using a scalpel, scissors, and radio frequency energy. Minimally invasive procedures in soft tissue such as the breast, however, are difficult to perform using standard scissors and scalpel. Furthermore, in a closed environment, radio frequency current dissipates into the surrounding tissue causing a decreased ability to achieve a current at the cutting electrode of sufficient high density to initiate a cut. To overcome this problem, high power settings are often required to initiate the cut which often is painful and increases thermal damage to the tissue whether using a standard or a custom electrosurgical generator.

Another problem associated with severing tissue is the control of bleeding. Radio frequency energy controls bleeding by coagulating small blood vessels. Another method of controlling bleeding is through the use of heat. For example, the Shaw Hemostatic Scalpel uses direct heat. However, while the bleeding is generally controlled, the cutting of tissue is often slower than with radio frequency energy and the knife edge readily dulls. The Harmonic Scalpel (Ethicon Endosurgery) uses ultrasonic energy generally at 50 kHz to heat the tissue so as to coagulate severed blood vessels but cuts slower than a standard electrosurgical electrode and is costly as a custom ultrasonic generator is required.

A further disadvantage of using radio frequency energy is the generation of smoke. The smoke is malodorous and can contain airborne viral particles that may be infectious. Furthermore, during laparoscopic procedures, the smoke generated within the abdominal cavity often obscures visualization of the procedure. When the smoke becomes too dense, the procedure is delayed until the smoke is released through one of the trocar ports and after enough carbon dioxide gas has reinsufflated the abdominal cavity. This unnecessarily prolongs the operative time.

Accordingly, there is a need for efficient severing or cutting of tissue preferably with the ability to control bleeding from small severed blood vessels that can be used during a minimally invasive procedure and/or during an open surgical procedure.

### SUMMARY OF THE INVENTION

Devices for efficient severing or cutting of a material or substance such as soft tissue suitable for use in open surgical and/or minimally invasive procedures are disclosed. It should be appreciated that the present invention can be implemented in numerous ways, including as a process, an apparatus, a system, a device, and a method. Several inventive embodiments of the present invention are described below.

A cutting assembly generally includes a first and a second cutting blade each having an inner surface, an outer surface opposite the inner surface, one or more cutting edges, and a set of cutting teeth disposed along at least a portion of the cutting edge, the cutting assembly being configured with the first inner surface opposing the second inner surface so that the first set of cutting teeth is aligned with and configured to cooperate with the second set of cutting teeth. The cutting blades are preferably serrated with multiple teeth on one or more edges.

A tissue cutting device generally includes a probe defining a probe axis, the cutting assembly configured to be in a storage configuration or a cutting configuration, and a cutting apparatus coupled to the cutting assembly to rotate and/or oscillate at least one of the cutting blades relative to the other when the cutting assembly is in the cutting configuration. The cutting apparatus may rotate/oscillate the first cutting blade while maintaining the second cutting blade stationary relative to the probe or may rotate/oscillate the first cutting blade and the second cutting blade in opposing directions. When the teeth of the first oscillating/rotating cutting blade approximates opposing teeth of the second cutting blade, material or tissue caught between the opposing teeth of the blades is sheared. The multiple teeth along both cutting blades define a multiple scissor array that severs or shears material or tissue along a length of the advancing cutting assembly. Where the cutting apparatus oscillates at least one of the cutting blades, the oscillation has a peak to peak distance of at least a distance between two adjacent cutting teeth on the first and/or second set of cutting teeth. The cutting teeth may include edge serrations. The cutting blades may provide cooperating blade alignment elements so as to align the first and second cutting blades relative to each other.

The cutting assembly may be at least partially retracted within the probe in the storage configuration and at least partially return to the cutting configuration when extended through a distal end of the probe. The probe may include a cover slidable between a proximal position in which the cutting assembly is at least partially in the cutting configuration and a distal position in which the cover at least partially houses the cutting assembly in the storage configuration. The probe may define one or more openings along a length in a distal region thereof from which the cutting assembly extends from the storage configuration to the cutting configuration in a direction generally orthogonal to a probe axis.

In one embodiment, the first and second cutting blades are generally circle arcs in shape and at least one of the first and second cutting blades is configured to pivot relative to the other about a cutting assembly pivot. In another embodiment, the first and second cutting blades form embedded cylinders such that the cutting assembly defines a cutting direction generally in alignment with the probe axis. In yet another embodiment, multiple cutting assemblies each defining a cutting axis generally orthogonal to the probe axis are coupled to each other circumferentially via a loop cable, the cutting assemblies and the loop cable being configured to be rotatable about the probe axis.

The cutting assembly may be configured as at least a partial loop attached to a loop holder defining a loop holder axis generally orthogonal to the probe axis, the loop generally returning to the cutting configuration from the storage configuration. The loop holder is configured to rotate the loop cutting assembly about the loop holder axis when the cutting assembly is in the cutting configuration so as to adjust a loop angle defined between the probe axis and the cutting assembly.

At least one of the cutting blades may be operatively coupled to an energy source selected from radio frequency, laser and ultrasonic energy, heat, cold, and air or liquid pressure. At least one of the cutting blades may be at least partially insulated.

A coagulator may be incorporated into the cutting assembly. For example, the coagulator may be disposed on the first and/or second outer surfaces of the cutting blades. The coagulator can be coupled to an energy source such as a radio frequency energy, laser, cold, ultrasonic heating, and/or electrical resistive heating source. The coagulator may be an inductive coil configured around at least a portion of at least one of the first and second cutting blades. An energy source may be coupled to the coagulator to deliver an electrical current through the inductive coil to cause at least part of the cutting assembly surrounded by the inductive coil to increase in temperature through inductive heating. A temperature sensor may also be incorporated into the cutting assembly to provide a feedback mechanism for controlling a temperature of at least one of the cutting blades and the coagulator. A tissue collector may be incorporated into at least one of the cutting assembly and the probe.

A method for cutting tissue generally includes positioning a distal region of a probe of a tissue cutting device adjacent to a region of tissue to be severed, the probe defining a probe axis, returning a cutting assembly to a cutting configuration from a storage configuration, moving at least one of a first cutting blade and a second cutting blade of the cutting assembly relative to the other, the moving being at least one of rotating and oscillating, the first cutting blade having a first inner surface, a first outer surface opposite the first inner surface, a first cutting edge, and a first set of cutting teeth disposed along at least a portion of the first cutting edge, the second cutting blade having a second inner surface, a second outer surface opposite the second inner surface, a second cutting edge, and a second set of cutting teeth disposed along at least a portion of the second cutting edge, the first inner surface being configured opposing the second inner surface so that the first set of cutting teeth is aligned with and configured to cooperate with the second set of cutting teeth, and one of advancing and retracting the tissue cutting device during the moving of at least one of the cutting blades such that the cutting assembly severs the tissue.

These and other features and advantages of the present invention will be presented in more detail in the following detailed description and the accompanying figures which illustrate by way of example principles of the invention.

### BRIEF DESCRIPTION OF THE DRAWINGS

The present invention will be readily understood by the following detailed description in conjunction with the accompanying drawings, wherein like reference numerals designate like structural elements.

FIGS. 1A and 1B are perspective views of exemplary embodiments of a cutting assembly.

FIGS. 2A, 2B, 2D and 2E are perspective views of exemplary embodiments of a cutting blade.

FIG. 2C is a cross-sectional top view of a tooth taken through plane A in FIG. 2A.

FIGS. 3A-1 to 3D-2 are perspective views of a cutting assembly illustrating a mechanism of cutting.

FIGS. 4A and 4B are perspective views of further exemplary embodiments of teeth on the cutting blade.

FIG. 4C is a perspective view of an exemplary embodiment of a cutting apparatus.

FIGS. 5A-5C are perspective views of a cutting blade with exemplary embodiments of coagulators.

FIGS. 6A-6D are perspective views of exemplary embodiments of cutting devices.

FIG. 7 is a perspective view of an exemplary embodiment of a cutting device.

FIGS. 8A and 8B are perspective views of an exemplary embodiment of a cutting device.

FIGS. 9A-9C are a perspective, a side, and a cross-sectional top view, respectively, of an exemplary embodiment of a cutting device.

FIGS. 10A-10D are perspective views of an exemplary embodiment of a cutting device.

### DESCRIPTION OF SPECIFIC EMBODIMENTS

Devices for efficient severing or cutting of a material or substance such as soft tissue suitable for use in open surgical and/or minimally invasive procedures are disclosed. The following description is presented to enable any person skilled in the art to make and use the invention. Descriptions of specific embodiments and applications are provided only as examples and various modifications will be readily apparent to those skilled in the art. The general principles defined herein may be applied to other embodiments and applications without departing from the spirit and scope of the invention. Thus, the present invention is to be accorded the widest scope encompassing numerous alternatives, modifications and equivalents consistent with the principles and features disclosed herein. For purpose of clarity, details relating to technical material that is known in the technical fields related to the invention have not been described in detail so as not to unnecessarily obscure the present invention.

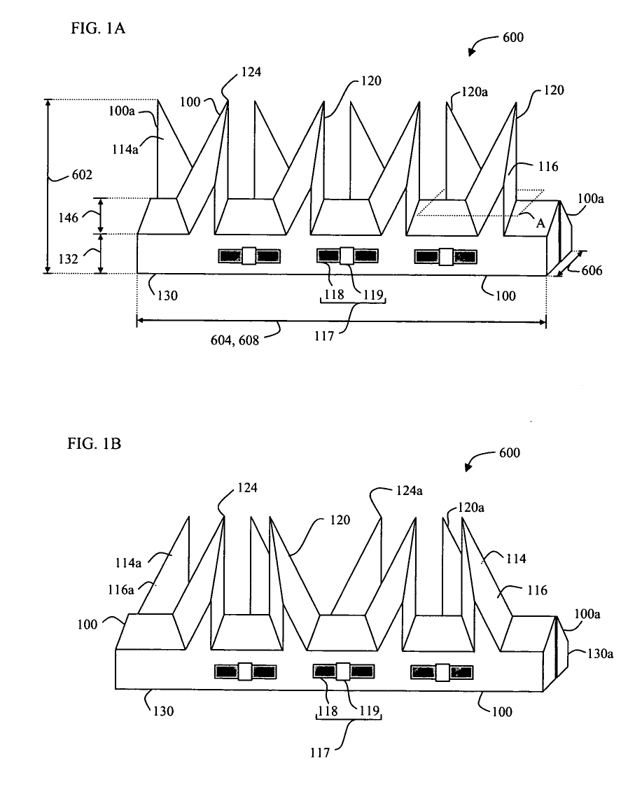

Exemplary embodiments of a cutting assembly 600 are illustrated in FIGS. 1A and 1B. The cutting assembly 600 includes cutting blades 100, 100a. Cutting blades 100, 100a have inner surfaces 114, 114a and outer surfaces 116, 116a. Preferably the inner surfaces 114, 114a are planar and the outer surfaces 116, 116a are beveled. The cutting assembly 600 is configured such that the inner surfaces 114, 114a are opposing. The cutting assembly 600 has a cutting assembly height 602, a cutting assembly length 604 and a cutting assembly width 606. The cutting assembly length 604 defines a cutting assembly axis 608.

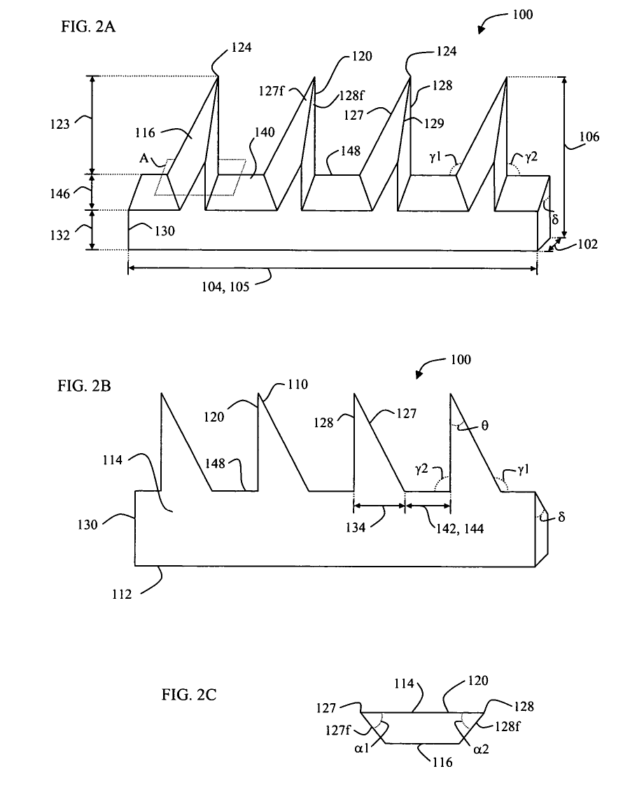

An exemplary embodiment of the cutting blade 100 is illustrated in FIGS. 2A and 2B. Perspective views of the outer surface 116 and the inner surface 114 are illustrated in FIG. 2A and FIG. 2B, respectively. Further exemplary embodiments of the cutting blade 100 are illustrated in FIGS. 2D and 2E. The cutting blades 100, 100a may be configured to be identical or not identical, i.e., variable. The cutting blade 100 has a blade width 102, a blade length 104, a blade height 106, a leading edge 110 and a trailing edge 112. The blade length 104 defines a blade axis 105. The blade width 102 and/or the blade height 106 can be constant or variable and variations in the blade width 102 and/or the blade height 106 can be symmetric or asymmetric. The cutting blade 100 preferably includes one or more teeth 120 on the leading edge 110 and/or the trailing edge 112, one or more valleys 140 on the leading edge 100 and/or the trailing edge 112 and a base 130. The base 130 has a base height 132. The base height 132 can be constant or variable. Preferably, the base height 132 and/or the blade width 102 is of sufficient strength to prevent deformation of the cutting loop 100 during cutting. The tooth 120 defines a tooth height 123, tooth edges 127, 128, 129, bevel surfaces 127f, 128f, and a tooth angle θ. The distance between tooth peaks 124 of similarly configured teeth 120 defines a pitch 126 as shown in FIGS. 2D and 2E. Preferably, the pitch 126 is in the range of 0.5 to 1.5 millimeters although the pitch 126 may be less than 0.5 mm or greater than 1.5 mm. The pitch 126 can be identical or may vary along cutting blade 100.

A convergence of tooth edges 127, 128, 129 defines a tooth peak 124 which is shown configured as a tip. The relation of bevel surface 127f to the inner surface 114 defines a bevel angle α1 and the relation of bevel surface 128f to the inner surface 114 defines a bevel angle α2 as shown in FIG. 2C, which is a cross-sectional top view of the tooth 120 taken through plane A in FIG. 2A. Each of bevel angles α1 and α2 is preferably approximately 45 to 75°. A bevel angle al or α2 of less than 45° may weaken the cutting blade 100 and a bevel angle α1 or α2 greater than 75° may decrease the sharpness of the tooth peak 124 and tooth edges 127, 128. On each tooth 120, bevel angles α1, α2 can be equal or unequal. Furthermore, the one or more teeth 120 configured on cutting blade 100 have a configuration of bevel angles α1, α2 that is the same or different and symmetric or asymmetric. In an alternative (not shown), the tooth peak 124 defines a tooth peak width and a tooth peak length. The tooth peak width is preferably less than the blade width 102 but may also be greater than or equal to the blade width 102. The tooth peak length may vary from a tip to the entire length of the cutting blade 100.

The valley 140 defines a valley edge 148 and a valley height 146. The valley edge 148 defines a valley angle δ. The valley angle δ is preferably 45° to increase the sharpness of the valley edge 148. The valley edge 148 can be linear, curved, faceted, serrated and/or regular or irregular. The valley edge 148 defines a valley edge length 142. The valley edge length 142 can range from 0 to almost the cutting blade length 104. The valley edge lengths 142 along the cutting blade 100 can be symmetric or asymmetric. A width of the tooth 120 at the valley edge 148 defines a tooth base width 134.

Tooth edges 127, 128 in relation to adjacent valley edges 148 define tooth edge angles γ1 and γ2. In the embodiment illustrated in FIGS. 2A and 2B, tooth edge angle γ2 is generally 90° and tooth edge angle γ1 is greater than 90. The tooth edge angles γ1 and γ2 can be equal or unequal. Furthermore, the configuration of tooth edge angles γ1 and γ2 on each tooth 120 on the cutting blade 100 may be the same or different and/or symmetric or asymmetric. Tooth edges 127, 128, 129 may be, for example, linear, curved, faceted, serrated, sharpened, irregular, and/or symmetric or asymmetric.

In curved tooth edges 127, 128 as shown in FIG. 2D, not all the parameters described herein are readily definable (for example, valley edge length 142). Although the teeth 120 are illustrated as identical and symmetrically positioned on the cutting blade 100 in FIGS. 2A-2C, the teeth 120 may alternatively be not identical and/or asymmetrically positioned on the cutting blade 100.

The cutting blade 100 may be formed from a metal, a metal alloy, glass, mineral, ceramic, plastic and/or a polymer. The cutting blade 100 may be rigid or flexible. A flexible cutting blade 100 is preferably configured from a material having sufficient elastic properties to prevent a significant permanent deformity when external stresses are placed on the flexible cutting blade 100 when the external stresses do not exceed the strain limits of the material of the flexible cutting blade 100. Furthermore, the material of the cutting blade 100 preferably has sufficient strength to prevent deformation of the cutting blade 100 during the cutting procedure. The cutting blade 100 is configured using techniques and methods well known to those skilled in the art and may include machining, lasering, stamping, and/or chemical etching. In a further embodiment, the cutting blade 100 may include multiple materials. The multiple materials can be configured as one or more layers, segments and/or portions that are continuous or discontinuous and symmetric or asymmetric. The multiple material provide properties such as electrical insulation, heat insulation, varying conductivity (for example, heat or electricity), increased hardness, lubricity, and/or sensors (for example, temperature). Materials configured as surface coatings on the cutting blade 100 may include polymers, plastics, ceramics, diamond-like carbon, diamond and/or diamond-like noncomposite coatings (metal-doped and nonmetal-doped). One or more liquid materials may also be incorporated into the cutting assembly 600 to facilitate, for example, lubricity or heat insulation. Such materials include, for example, silicone and perfluorinated fluids.

Referring again to FIGS. 1A and 1B, at least one of the cutting blades 100, 100a oscillates, preferably along the cutting assembly axis 608. A peak to peak distance of oscillation and/or a frequency of oscillation may be predetermined or variable. The peak to peak distance is preferably at least as long as the sum of adjacent tooth base width 134 and valley edge length 142 as shown in FIG. 2B, e.g., the distance between adjacent teeth 120, such that each tooth 120 passes at least one tooth 120a during movement in a single direction or one-half of a cycle of oscillation. The frequency of oscillation is preferably between 50 and 100 Hz but can also be less than 50 Hz or greater than 100 Hz. In an alternative, the cutting blades 100, 100a both oscillate in opposing directions.

To maintain the cutting blades 100, 100a in close apposition, one or more blade aligners 117 may be provided, preferably on bases 130, 130a. The blade aligners 117 may define slots 118 and slot fasteners 119. The slot fasteners 119 pass through at least adjacent slots 118 of the cutting blades 100, 100a. The slots 118 allow at least one of the cutting blades 100, 100a to oscillate relative to the other while the slot fasteners 119 keep the cutting blades 100, 100a in close approximation. Another embodiment of a blade aligner 117 includes grooves (not shown) on the bases 130, 130a of the cutting blades 100, 100a that interconnect with each other. Various other suitable mechanisms for keeping the cutting blades 100, 100a in close approximation may be alternatively or additionally utilized.

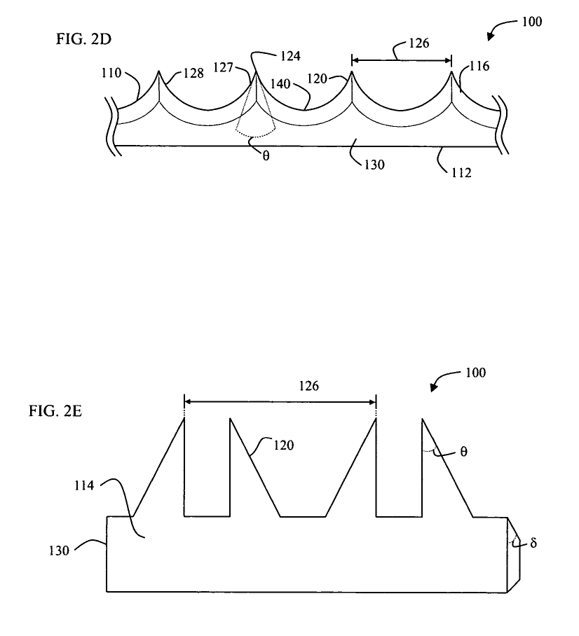

FIGS. 3A-1 to 3D-2 illustrate an exemplary mechanism of cutting using the cutting assembly 600. FIGS. 3A-1, 3B-1, 3C-1 and 3D-1 are cross-sectional top views taken through a plane A in FIGS. 3A-2, 3B-2, 3C-2 and 3D-2, respectively. The cutting assembly 600 automatically advances or preferably is manually advanced to cut in a direction 620. The cutting blade 100a oscillates along opposing oscillation directions 630, 631 which are preferably along the cutting assembly axis 108. In the example shown, the cutting blade 100 does not oscillate. As shown in FIG. 3B-1, a shearing force occurs between opposing teeth 120, 120a at intersections 610 during movement of the cutting loop 100a in oscillation direction 630. The cutting assembly 600 cuts along a plane C as shown in FIG. 3C-2 when the cutting assembly 600 is advanced in direction 620. The multiple shearing effects or multiple scissor array of the teeth 120a against the teeth 120 creates multiple scissor-like cuts along the path of advancing the cutting assembly 600. Furthermore, the nonoscillating cutting blade 100 at least partially fixates and reduces the substance or material being cut from moving in directions 630, 631 with the oscillating cutting blade 100a by partially embedding tooth peaks 124 into the substance or material. Stabilization of the substance or material being cut facilitates cutting. Movement of the substance or material during cutting deters the cutting process by preventing engagement of the material between adjacent teeth 120, 120a prior to shearing of the material.

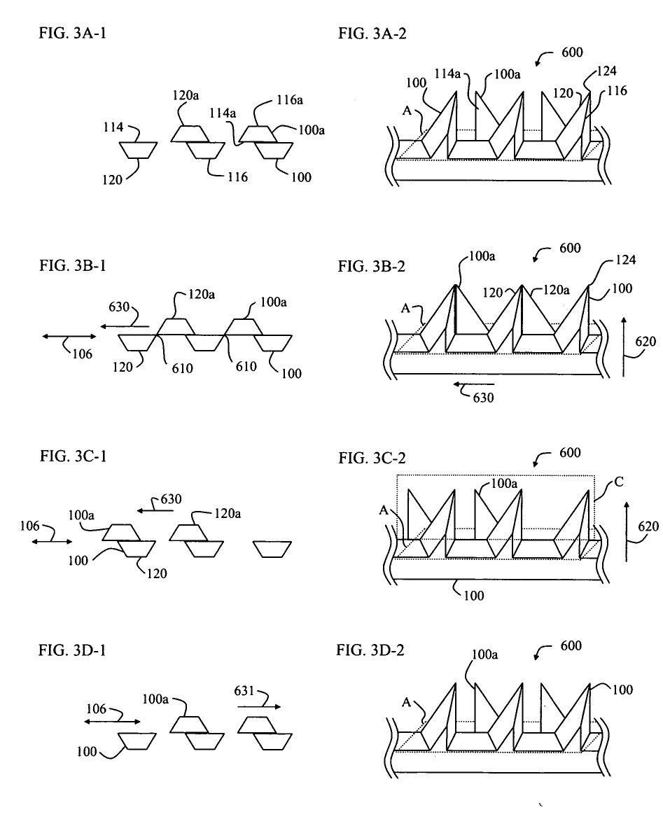

In further exemplary embodiment as illustrated in FIGS. 4A and 4B, tooth edges 127, 128 may include one or more edge serrations 121. In the cutting assembly 600 shown in FIG. 4C, the edge serrations 121, 121 a facilitate fixation of tissue between adjacent teeth 120, 120a on cutting blades 100, 100a by preventing tissue or other material being cut between the adjacent teeth 120, 120a from moving in the direction 620 as tooth 120 approximates adjacent tooth 120a during oscillation of the cutting blade 100a in direction 630.

In an alternative embodiment (not shown), cutting blade 100 and cutting blade 100a oscillate in opposing directions. Cutting blades 100, 100a oscillating in opposing directions reduces movement of the material being cut by preventing the material from moving in a single direction at a given moment in time. In another alternative embodiment (not shown), the cutting assembly 600 may include one or more cutting blades that rotate. A nonrotating cutting blade or a secondary cutting blade rotating in an opposing direction facilitates fixation of the material being cut by preventing the material from moving in the direction of the primary rotating cutting blade.

In a further embodiment, the cutting assembly has three or more cutting blades. For example, a central cutting blade may oscillate while two outer cutting blades do not oscillate. Alternatively, the central cutting blade does not oscillate while the two outer cutting blades oscillate, preferably in opposing directions. Various other suitable combinations of cutting blade configuration, cutting assembly configuration, oscillation and/or rotation may be employed.

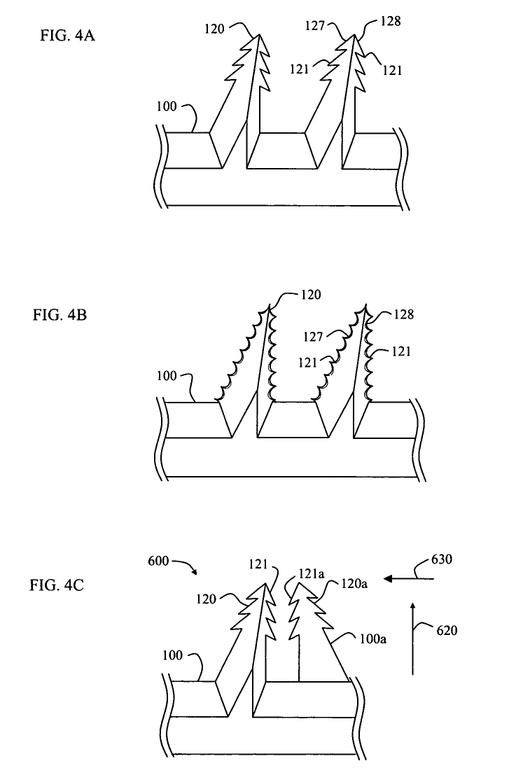

In a further exemplary embodiment as shown in FIGS. 5A-5C, at least part of the cutting blade 100 is configured to coagulate blood vessels to decrease bleeding during severing of living tissue. Preferably at least part of the base 130 is configured to coagulate blood vessels. Alternatively or additionally, a coagulator 700 may be provided on the outer surface 116 (as shown in FIG. 5A) and/or the trailing edge 112 of the cutting blade 100. The cutting blade 100 and/or the coagulator 700 may be operatively coupled to an energy source. The energy source may be a radio frequency energy, laser, cold, ultrasonic heating, and/or electrical resistive heating. Cutting blades 100, 100a may be insulated, partially insulated or not insulated. Heat facilitates coagulation of blood vessels by denaturing proteins which decreases bleeding during cutting of living tissue. Preferably the tissue is heated in a range of 50 to 100° C. The energy source may be external to or incorporated into a device that includes the cutting assembly 600.

In an alternative as shown in FIGS. 5B and 5C, an inductive coil 702 is configured around at least part of the base 130. An electrical current from an energy source may pass through the inductive coil 702 to cause at least part of the base 130 surrounded by the inductive coil 702 to increase in temperature through inductive heating. As shown in FIG. 5B, the inductive coil 702 passes through inductive slots 704 located in the base 130. In an alternative, as shown in FIG. 5C, inductive channels 720 facilitate wrapping of the inductive coil 702 around at least part of the base 130 during manufacturing of the cutting blade 100. In another alternative (not shown), the inductive coil 702 may pass through adjacent inductive channels 720 on the cutting blades 100, 100a in the cutting assembly 600. In this configuration, the inductive coil 702 may also act as slot fasteners 119 to keep the cutting blades 100, 100a in close approximation during oscillation. In a further embodiment, one or more temperature sensors may be incorporated into the cutting assembly 600 as part of a feedback mechanism to control the temperature of the cutting blades 100, 100a, the coagulator 700, and/or the bases 130, 130a and/or control the electrical current passing through the inductive coil 702.

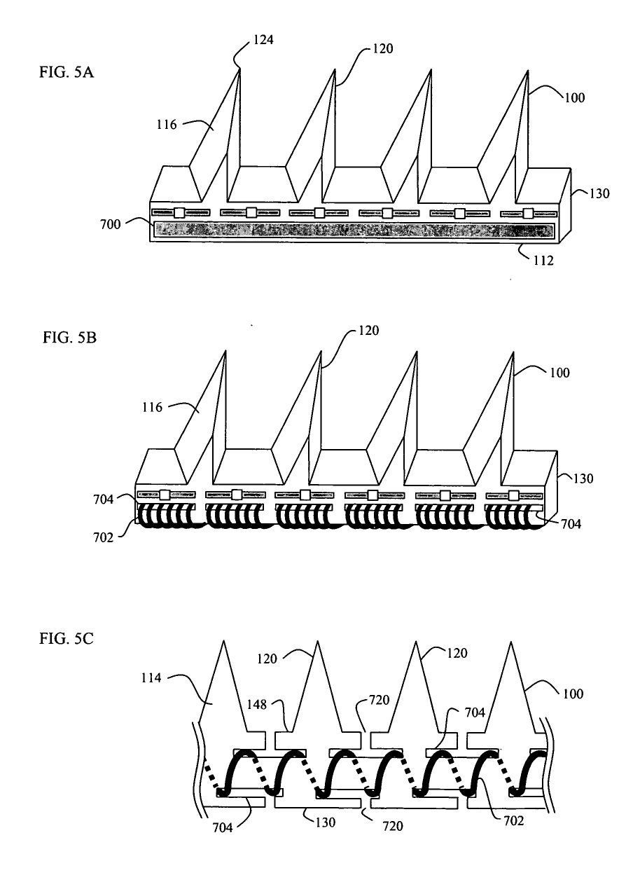

Various exemplary embodiments of a tissue cutting device 800 including the cutting assembly 600, a probe 820 and a handle 840 are illustrated in FIGS. 6-10. The probe 820 has a length that defines a probe axis 822. The probe 820 may be linear, angled, and/or curved. The cut direction 620 is created by advancing along, moving orthogonal to and/or rotating around the probe axis 822. At least one of the cutting blades 100, 100a preferably oscillates along or orthogonal to the cutting assembly axis 608 and is operatively connected to an oscillator (not shown) located in the handle 840 and/or the probe 820. The oscillator is controlled by an oscillating controller (not shown) which may be located on the handle 840 or as a foot control. The oscillator may be powered by alternating or direct current, vacuum, gas pressure and/or liquid pressure. When direct current is used, one or more batteries may be located within or external to the handle 840.

When one or more external energy sources are used, one or more energy couplers 846 extend from the handle 840 to the one or more external energy sources. For example, when radio frequency energy is incorporated, an external electrosurgical generator is operatively coupled to the tissue cutting device 800 using the energy coupler 846. When incorporating radio frequency energy, the tissue cutting device 800 may be a monopolar or a bipolar system.

The cutting assembly 600 is preferably housed in a sheath or probe cover 830 and/or the probe 820 when not oscillating or cutting. The cutting assembly 600 is exposed by advancing through a distal end 823 of the probe 820 and/or by retracting the probe cover 830 prior to, simultaneously with, or during activation of the oscillation. A cutting assembly advancer 844 is located on the handle 840 when the cutting assembly 600 is housed in the probe 820. Control of retraction of the probe cover 830 or advancement of the cutting assembly 600 through the distal end 823 and oscillation of the cutting assembly 600 may be separate or combined. In one alternative, a safety mechanism operatively couples retraction of the probe cover 830 with activation of oscillation. Activation of a safety controller (not shown) located on the handle 840, retracts the probe cover 830 and activates the oscillation. When the safety controller is deactivated, the oscillation stops and the probe cover 830 advances over the cutting assembly 600. The safety controller may be in any suitable configuration and may include, for example, one or more buttons, triggers, levers, knobs, and/or pedals.

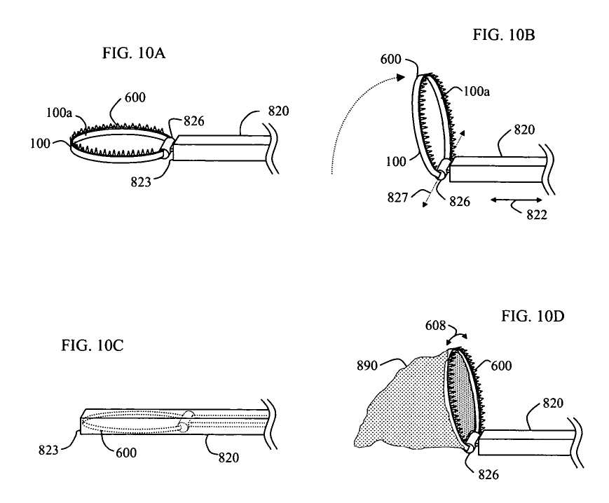

Various additional components may be incorporated in the tissue cutting device 800. For example, a tissue collector (such as a tissue collector 890 shown in FIG. 10D) may be attached to the cutting assembly 600 and/or the probe 820. A tissue marker may be attached to the cutting assembly 600, the tissue collector 890 and/or the probe 820. A tissue penetrator may be positioned at or near the distal end 823. In addition, an imaging, tracking or locating device may be incorporated into the tissue cutting device 800. As yet another example, the tissue cutting device 800 may include one or more channels for evacuation of fluids and/or material from the cutting area and/or for instillation of liquid(s) and/or other substance(s) into the cutting area. The one or more channels may be operatively connected to a vacuum source.

In FIG. 6A, the cutting assembly axis 608 is generally aligned with the probe axis 822. Oscillation of at least one of the cutting blades 100, 100a is along the cutting assembly axis 608 and the direction of cut 620 is orthogonal to the probe axis 822. A length of exposed cutting assembly 612 may be fixed or variable. The cutting assembly advancer 844 preferably manually controls the length of exposed cutting assembly 612. As shown in FIG. 6B, the length of exposed cutting assembly 612 is longer than in FIG. 6A. In FIG. 6C, the cutting assembly 600 is housed in the sheath 830. In a further embodiment (not shown), multiple cutting assemblies 600 and/or probes 820 attachable to the handle 840 may be provided such as in a kit, such that one of the cutting assemblies 600 and/or probe 820 may be selectively attached to the handle 840. In yet further embodiments (not shown), the configuration of the exposed cutting assembly 612 may be curved, angled and/or irregular or regular.

In the exemplary embodiment illustrated in FIGS. 6C and 6D, oscillation of at least one of the cutting blades 100, 100a is generally orthogonal to the cutting assembly axis 608 and the cut direction 620 is along the probe axis 822. Various suitable mechanisms of oscillation of at least one of the cutting blades 100, 100a may be employed as is well known in the art. For example, at least one of cutting blades 100, 100a may oscillate about a cutting assembly pivot 860. In a further embodiment as shown in FIG. 6D, when the cutting assembly 600 is exposed by advancing through the distal end 823 and/or retracting the sheath or probe cover 830, a width of cut 640 by the cutting assembly 600 is greater than a probe width 836 and/or a probe cover width 832. The cutting blades 100, 100a may generally be in a shape of circle sectors and the teeth on the cutting blades 110, 100a form the circular arcs of the circle sectors. One or both cutting blades 100, 100a oscillate or rotate. When both cutting blades 100, 100a oscillate or rotate, the oscillation or rotation is in opposing directions.

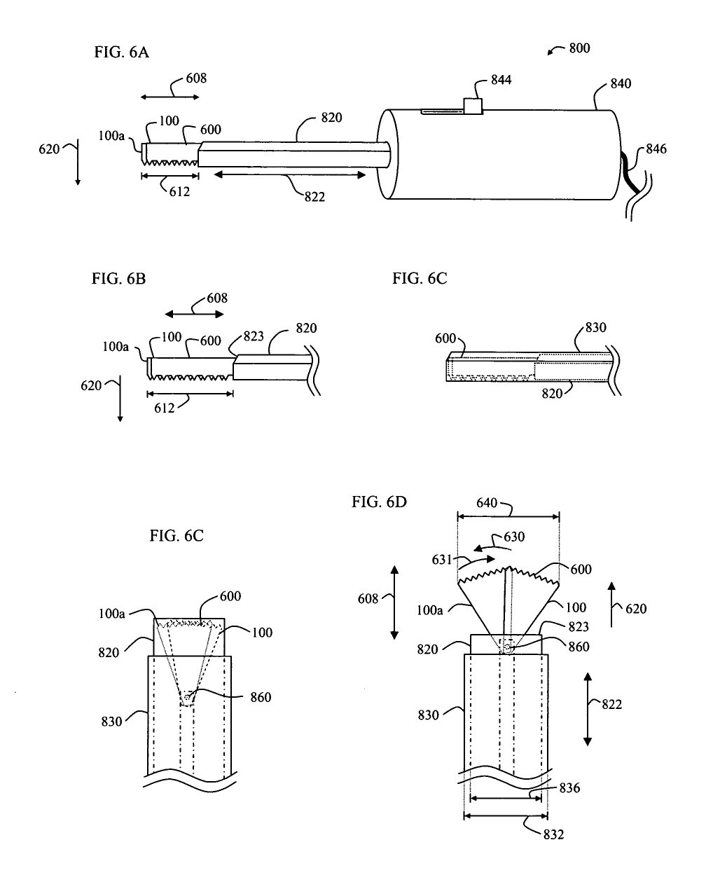

In a further exemplary embodiment illustrated in FIG. 7, the cutting assembly 600 is configured as a core or cylinder 750 in which an inner cylindrical cutting blade 100a abuts and nests within an outer cylindrical cutting blade 100. The core 750 advances in the cut direction 620 to cut a core, i.e., cylinder, of material or tissue. Severing is achieved by oscillation or rotation of one of the cutting blades 100, 100a or both of the cutting blades 100, 100a in opposite directions.

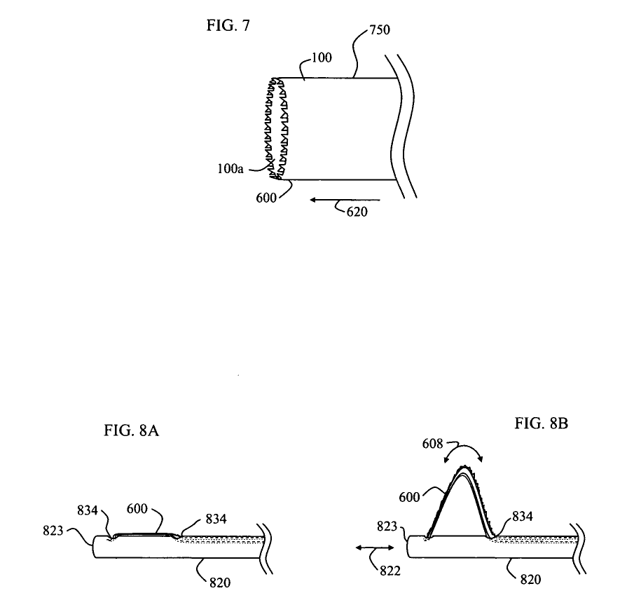

In the exemplary embodiments illustrated in FIGS. 8-10, the cutting assembly 600 is flexible. In FIGS. 8A and 8B, the cutting assembly 600 is extendable and retractable through one or more probe openings 834 located at or near the distal end 823. FIG. 8A shows the cutting assembly 600 in a retracted configuration and FIG. 8B shows the cutting assembly 600 in an extendable configuration. Cutting is achieved by moving the probe 820 in a direction generally orthogonal to the probe axis and/or by rotating the probe 820 about the probe axis 822.

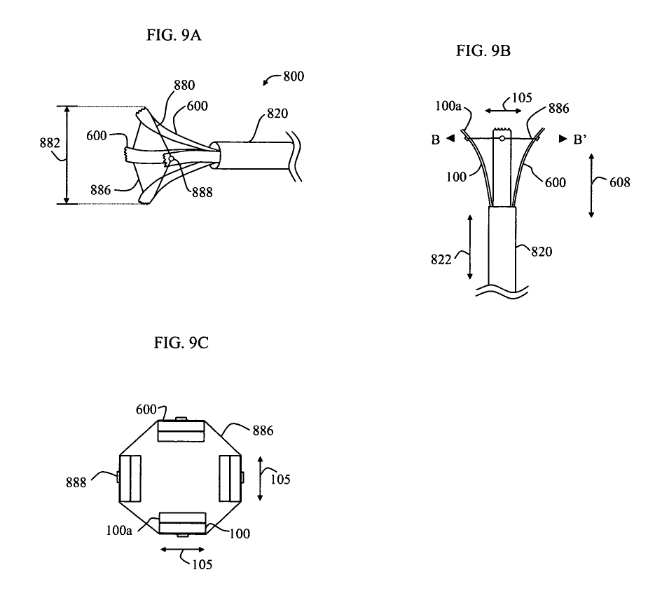

In FIGS. 9A-9C, a cutting loop 880 includes multiple cutting assemblies 600 and a loop cable 886. A cutting loop diameter 882 of the cutting loop 880 may be fixed or expandable. The expandable cutting loop 880 is preferably housed in a sheath or probe cover (not shown) and/or the probe 820 when not in use or during penetration of the tissue cutting device 800 (i.e., into tissue). The cutting assemblies 600 are preferably configured from a material with shape memory properties (for example, a nickel titanium alloy). The cutting assemblies 600 can be preformed to a predetermined shape as shown in a perspective view in FIG. 9A and in a side view in FIG. 9B such that arrangement of the cutting assemblies 600 when not within the confines of the probe 820 or sheath, determines a shape and the cutting loop diameter 882 of the cutting loop 880. The loop cable 886 may be affixed to or slidable through cable fasteners 888 that are positioned on the cutting assemblies 600. A length of the loop cable 886 determines the maximum diameter of the cutting loop 880. As shown in FIG. 9B and a cross-sectional top view in FIG. 9C taken through plane B-B′ in FIG. 9B, at least one of the cutting blades 100, 100a of each cutting assembly 600 oscillates along the cutting blade axes 105 which are generally orthogonal to the cutting assembly axis 608. In a further alternative, at least one of the cutting assemblies 600 and/or the loop cable 886 is energized preferably with radio frequency energy. In yet a further alternative, the cutting assemblies 600 and the loop cable 886 rotate around the probe axis 822 with or without rotation of the probe 820 in addition to oscillation of at least one of the cutting blades 100, 100a of the cutting assemblies 600 to facilitate cutting.

In a further exemplary embodiment as shown in FIGS. 10A-10D, the cutting assembly 600, which may be generally circular, elliptical or ovoid in shape, is mounted on a loop holder 826. The loop holder 826 is rotatable around a loop holder axis 827 that is generally orthogonal to the probe axis 822. When not activated, the cutting assembly 600 is housable in a storage configuration in the probe 820 as shown in FIG. 10C and/or in the probe cover (not shown). The cutting assembly 600 is exposed by one of advancing through the distal end 823 and retracting the probe cover. Cutting blades 100, 100a of the cutting assembly 600 are preferably configured from a material with a sufficiently high elastic property or superelastic property that upon application of one or more external stresses (i.e., the inner walls of the probe 820 or the probe cover), the elastic or superelastic property of the material allows the cutting blades 100, 100a to configure to the storage configuration without the development of a significant permanent deformity as long as the resulting strains do not exceed the recoverable strain limits of the material. When the cutting assembly 600 is sufficiently freed from the one or more external stresses (i.e. by advancing through the distal end 823 and/or by retracting the probe cover), the cutting assembly 600 returns to a predetermined cutting configuration. Oscillation and/or rotation of the cutting loops 100 and/or 100a is along the cutting assembly axis 608 and is activated when at least part of the cutting assembly 600 is exposed.

While the exemplary embodiments of the present invention are described and illustrated herein, it will be appreciated that they are merely illustrative and that modifications can be made to these embodiments without departing from the spirit and scope of the invention. Thus, the scope of the invention is intended to be defined only in terms of the following claims as may be amended, with each claim being expressly incorporated into this Description of Specific Embodiments as an embodiment of the invention.

1. A tissue cutting device, comprising:
a probe defining a probe axis; and
a cutting assembly configured to be in one of a storage configuration and a cutting configuration including:
a first cutting blade having a first inner surface, a first cutting edge, and a first set of cutting teeth disposed along at least a portion of the first cutting edge: and
a second cutting blade having a second inner surface, a second cutting edge, and a second set of cutting teeth disposed along at least a portion of the second cutting edge, the cutting assembly being configured with the first inner surface being in contact with and in close apposition with the second inner surface so that the first set of cutting teeth is aligned with and configured to cooperate with the second set of cutting teeth when at least one of the first and second cutting blades moves relative to the other when the cutting assembly is in the cutting configuration.

2. The tissue cutting device of claim 1, further comprising a cutting apparatus coupled to the cutting assembly, the cutting apparatus being configured to at least one of rotate and oscillate at least one of the cutting blades relative to the other, the oscillation having a peak to peak distance of at least a distance between two adjacent cutting teeth on at least one of the first set of cutting teeth and the second set of cutting teeth.

3. The tissue cutting device of claim 2, wherein the cutting apparatus is configured to at least one of rotate and oscillate the first cutting blade and wherein the second cutting blade is stationary relative to the probe.

4. The tissue cutting device of claim 2, wherein the cutting apparatus is configured to at least one of rotate and oscillate the cutting blades relative to each other in opposing directions.

5. The tissue cutting device of claim 1, wherein at least one of the first cutting blade and the second cutting blade has more than one cutting edge and more than one set of cutting teeth.

6. The tissue cutting device of claim 1, wherein the cutting teeth of at least a portion of at least one of the sets of cutting teeth includes edge serrations.

7. The tissue cutting device of claim 1, wherein each of the cutting blades has at least one of high elasticity, shape memory property and superelastic property.

8. The tissue cutting device of claim 1, wherein at least one of the cutting blades is coupled to an energy source selected from radio frequency, laser and ultrasonic energy, heat, cold, and air or liquid pressure.

9. The tissue cutting device of claim 8, wherein at least one of the cutting blades is at least partially insulated.

10. The tissue cutting device of claim 1, wherein the first cutting blade provides a first blade aligner and the second cutting blade provides a second blade aligner configured to cooperate with the first blade aligner to align the first and second cutting blades relative to each other.

11. The tissue cutting device of claim 1, wherein the cutting assembly is configured to be in the storage configuration when retracted into the probe and is at least partially in the cutting configuration when extended from a distal end of the probe.

12. The tissue cutting device of claim 1, wherein the probe includes a cover slidable between a proximal and a distal position, wherein when the cover is in the distal position, the cover at least partially houses the cutting assembly in the storage configuration and when the cover is in the proximal position, the cutting assembly is at least partially in the cutting configuration.

13. The tissue cutting device of claim 1, wherein the cutting assembly generally extends along the probe axis.

14. The tissue cutting device of claim 1, wherein the first and second cutting blades are generally circle sectors in shape and at least one of the first and second cutting blades is configured to pivot relative to the other about a cutting assembly pivot.

15. The tissue cutting device of claim 1, wherein the first cutting blade forms an outer cylinder and the second cutting blade forms an inner cylinder abutting and nesting within the first cutting blade, wherein the cutting assembly defines a cutting direction generally in alignment with the probe axis.

16. The tissue cutting device of claim 1, wherein the cutting assembly is configured as at least part of a loop, the device further comprising:
a loop holder defining a loop holder axis generally orthogonal to the probe axis, the loop holder being configured to hold and to rotate the cutting assembly about the loop holder axis when the cutting assembly is in the cutting configuration so as to adjust a loop angle defined between the probe axis and the cutting assembly.

17. The tissue cutting device of claim 1, wherein the probe defines at least one opening along a length of the probe in a distal region thereof from which the cutting assembly extends from the storage configuration to the cutting configuration in a direction generally orthogonal to the probe axis.

18. The tissue cutting device of claim 1, comprising a plurality of the cutting assemblies each defining a cutting axis generally orthogonal to the probe axis, the device further comprising:
a loop cable coupled to the plurality of the cutting assemblies, the cutting assemblies being disposed circumferentially about the loop cable, the plurality of the cutting assemblies and the loop cable and the plurality of the cutting assemblies being configured to be rotatable about the probe axis.

19. The tissue cutting device of claim 1, wherein the cutting direction is generally one of orthogonal to the probe axis, parallel to the probe axis and rotational around the probe axis.

20. The tissue cutting device of claim 1, further comprising a tissue collector coupled to at least one of the cutting assembly and the probe.

21. The tissue cutting device of claim 1, further comprising a coagulator integrated with the cutting assembly.

22. The tissue cutting device of claim 21, wherein the coagulator is disposed on at least one of the first and second outer surfaces of the cutting blades.

23. The tissue cutting device of claim 21, wherein the coagulator is coupled to an energy source selected from a radio frequency energy, laser, cold, ultrasonic heating, and electrical resistive heating source.

24. The tissue cutting device of claim 21, wherein the coagulator is an inductive coil configured around at least a portion of at least one of the first and second cutting blades.

25. The tissue cutting device of claim 24, further comprising an energy source coupled to the coagulator and configured to deliver an electrical current through the inductive coil to cause at least part of the cutting assembly surrounded by the inductive coil to increase in temperature through inductive heating.

26. The tissue cutting device of claim 21, further comprising a temperature sensor incorporated into the cutting assembly to provide a feedback mechanism for controlling a temperature of at least one of the cutting blades and the coagulator.

27. The tissue cutting device of claim 1, wherein the probe and the cutting assembly are configured as a first unit attachable to a handle.

28. The tissue cutting device of claim 27, further comprising at least one additional attachable units each being selectively attachable to the handle.

29. A method for cutting tissue, comprising:
positioning a distal region of a probe of a tissue cutting device adjacent to a region of tissue to be severed, the probe defining a probe axis;
returning a cutting assembly from a storage configuration to a cutting configuration;
moving at least one of a first cutting blade and a second cutting blade of the cutting assembly relative to the other, the moving being at least one of rotating and oscillating, the first cutting blade having a first inner surface, a first cutting edge, and a first set of cutting teeth disposed along at least a portion of the first cutting edge, the second cutting blade having a second inner surface, a second cutting edge, and a second set of cutting teeth disposed along at least a portion of the second cutting edge, the first inner surface being in contact and in close apposition with the second inner surface so that the first set of cutting teeth is aligned with and configured to cooperate with the second set of cutting teeth when at least one of the first and second cutting blades moves relative to the other; and
one of advancing and retracting the tissue cutting device during the moving of the cutting blades such that the cutting assembly severs the tissue.

30. The method for cutting tissue of claim 29, wherein the first and second cutting blades have more than one cutting edge and more than one set of cutting teeth, wherein the advancing and retracting of the tissue cutting device severs the tissue.

31. The method for cutting tissue of claim 29, wherein the cutting teeth of at least a portion of at least one of the sets of cutting teeth includes edge serrations.

32. The method for cutting tissue of claim 29, wherein the moving is at least one of rotating and oscillating the first cutting blade, the second cutting blade being stationary relative to the probe, and the oscillating having a peak to peak distance of at least a distance between two adjacent cutting teeth on at least one of the first set of cutting teeth and the second set of cutting teeth.

33. The method for cutting tissue of claim 29, wherein the moving includes one of rotating and oscillating the cutting blades relative to each other in opposing directions.

34. The method for cutting tissue of claim 29, wherein at least one of the first cutting blade and the second cutting blade has more than one cutting edge and more than one set of cutting teeth.

35. The method for cutting tissue of claim 29, wherein each of the cutting blades has at least one of high elasticity, shape memory property and superelastic property.

36. The method for cutting tissue of claim 29, wherein at least one of the cutting blades is coupled to an energy source selected from radio frequency, laser and ultrasonic energy, heat, cold, and air or liquid pressure.

37. The method for cutting tissue of claim 36, wherein at least one of the cutting blades is at least partially insulated.

38. The method for cutting tissue of claim 29, wherein the returning of the cutting assembly to at least partially the cutting configuration from the storage configuration includes at least extending the cutting assembly from a distal region of the probe.

39. The method for cutting tissue of claim 29, wherein the returning of the cutting assembly to at least partially the cutting configuration from the storage configuration includes sliding a probe cover from a distal to a proximal position, wherein when the probe cover is in the distal position, the probe cover at least partially houses the cutting assembly in the storage configuration and when the probe cover is in the proximal position, the cutting assembly is at least partially in the cutting configuration.

40. The method for cutting tissue of claim 29, wherein the cutting assembly generally extends along the probe axis.

41. The method for cutting tissue of claim 29, wherein the first and second cutting blades are generally circle sectors in shape and at least one of the first and second cutting blades is configured to pivot relative to the other about a cutting assembly pivot.

42. The method for cutting tissue of claim 29, wherein the moving at least one of the cutting blades relative to the other causes a circular cut in the tissue created by the first cutting blade forming an outer cylinder and the second cutting blade forming an inner cylinder abutting and nesting within the first cutting blade and wherein the advancing defines a cutting direction generally in alignment with the probe axis.

43. The method for cutting tissue of claim 29, wherein the returning includes extending the cutting assembly out of at least one opening defined along a length of the probe in a distal region thereof from the storage configuration to the cutting configuration in a direction generally orthogonal to the probe axis.

44. The method for cutting tissue of claim 29, further comprising:
rotating a plurality of the cutting assemblies each defining a cutting axis generally orthogonal to the probe axis and coupled to each other circumferentially via a loop cable.

45. The method for cutting tissue of claim 29, further comprising:
rotating a loop holder to rotate the cutting assembly configured as at least part of a loop attached to the loop holder about a loop holder axis defined by the loop holder, the loop holder axis being generally orthogonal to the probe axis, the rotating adjusts a loop angle defined between the probe axis and the cutting assembly.

46. The method for cutting tissue of claim 29, further comprising collecting the cut tissue with a tissue collector coupled to at least one of cutting assembly and the probe.

47. The method for cutting tissue of claim 29, wherein the cutting direction is generally one of orthogonal to the probe axis, parallel to the probe axis, and rotational around the probe axis.

48. The method for cutting tissue of claim 29, further comprising applying an energy to a coagulator integrated with the cutting assembly.

49. The method for cutting tissue of claim 48, wherein the energy is selected from the group consisting of radio frequency energy, laser, cold, ultrasonic heating, and electrical resistive heating.

50. The method for cutting tissue of claim 48, wherein the coagulator is an inductive coil configured around at least a portion of at least one of the first and second cutting blades.

51. The method for cutting tissue of claim 50, wherein the applying the energy includes delivering an electrical current through the coagulator, the coagulator being an inductive coil, and the applying the energy causing at least part of the cutting assembly surrounded by the inductive coil to increase in temperature through inductive heating.

52. The method for cutting tissue of claim 48, further comprising:
sensing a temperature in the cutting assembly and controlling a temperature of at least one of the cutting blades and the coagulator.

53. The method for cutting tissue of claim 29, wherein the probe and the cutting assembly are configured as a unit attachable to a handle, further comprising;
selecting an attachable unit from a plurality of the attachable units, each of the plurality of attachable units being selectively attachable to the handle; and
attaching the selected attachable unit to the handle.
